(19) United States
(12) Patent Application Publication
(10) Pub. No.: US 2023/0197252 A1
(43) Pub. Date: Jun. 22, 2023

(54) MEDICAL IMAGE PROCESSING SYSTEM, MEDICAL IMAGE PROCESSING METHOD, INFORMATION PROCESSING APPARATUS, AND PROGRAM

(71) Applicant: FUJIFILM Corporation, Tokyo (JP)

(72) Inventor: Daiki UEHARA, Tokyo (JP)

(73) Assignee: FUJIFILM Corporation, Tokyo (JP)

(21) Appl. No.: 18/168,571

(22) Filed: Feb. 14, 2023

Related U.S. Application Data
(63) Continuation of application No. PCT/JP2021/027899, filed on Jul. 28, 2021.

(30) Foreign Application Priority Data
Sep. 8, 2020 (JP) 2020-150287

(51) Int. Cl. G16H 30/40; G06T 7/00; G06T 1/60; G16H 30/20

(52) U.S. Cl. CPC G16H 30/40; G06T 7/0012; G06T 1/60; G16H 30/20; G06T 2207/30008; G06T 2207/30064; A61B 6/505

(57) ABSTRACT

A system, a method, an apparatus, and a medium that are related to a medial image processing or an information processing. An image processing server that performs a plurality of types of image processing receives and a processing request from an information processing apparatus, executes image processing corresponding to the processing request, and returns a processing result to a request source. The information processing apparatus collects an operating log, calculates priority of each of the plurality of types of image processing based on the collected operating log, and updates and manages priority information. The information processing apparatus acquires a new image, determines executable image processing for the acquired image, and transmits, based on the priority of each executable image processing and on a load situation of the image processing server, a processing request to the image processing server in accordance with a criterion of the priority.

### CROSS-REFERENCE TO RELATED APPLICATIONS

The present application is a Continuation of PCT International Application No. PCT/JP2021/027899 filed on Jul. 28, 2021 claiming priority under 35 U.S.C § 119(a) to Japanese Patent Application No. 2020-150287 filed on Sep. 8, 2020. Each of the above applications is hereby expressly incorporated by reference, in its entirety, into the present application.

### BACKGROUND OF THE INVENTION

### 1. Field of the Invention

The present invention relates to a medical image processing system, a medical image processing method, an information processing apparatus, and a program and particularly, to a medical image processing technology suitable for provision of a medical image processing service in which an image processing server receives an image of a processing target and a processing request, executes processing corresponding to the processing request, and returns a processing result to a request source.

### 2. Description of the Related Art

In the medical field, advances in image diagnostic apparatuses such as a computed tomography (CT) apparatus and a magnetic resonance imaging (MRI) apparatus have enabled image diagnosis using high-quality medical images having a high resolution. Particularly, in recent years, use of artificial intelligence (AI) by using a neural network trained by deep learning has improved accuracy of analysis processing for recognizing a lesion region or the like from an image or specifying a classification such as a disease name.

For example, the analysis processing such as computer aided diagnosis or computer aided detection (CAD) is generally prepared for each part such as a lung, a heart, a liver, and a brain or further, for each detectable lesion. In addition to CAD, an AI processing module for performing report creation support processing of automatically generating a candidate for a medical opinion in a radiographic interpretation report or the like has also been developed.

WO2020/158100A suggests a system for optimizing allocation of calculation resources such as a central processing unit (CPU) and a memory to various types of analysis processing within allocated calculation resources in an image processing program for performing various types of medical image analysis.

In addition, JP2005-218847A discloses a method of optimizing processing of archiving and/or communicating medical images captured using an image diagnostic apparatus in an image archiving and communication system (picture archiving and communication system (PACS)) of medical images.

### SUMMARY OF THE INVENTION

While a configuration of performing various types of analysis processing for medical images in a server or in a terminal present in a medical institution internal network can also be employed, a medical image processing service for receiving a medical image stored in each medical institution and a processing request from a plurality of medical institutions and returning a processing result to a request source using an image processing application programming interface (API) has been provided in recent years. For example, various medical image processing functions such as lung CAD are provided from an image processing API server. For example, lung CAD includes an AI processing module using a trained AI model that detects a lung disorder region and/or that outputs a recognition result and the like of a disorder name (disease name) using a CT image of a lung as input data.

In each medical institution using such a medical image processing service, shortening a time from imaging of an examination by a modality such as a CT apparatus to output of a medical image processing result to the highest extent possible is required in order to use processing results of various types of medical images in a diagnostic workflow such as a radiographic interpretation work as quickly as possible.

In order to handle such a request, a function of automatically acquiring an image from a digital imaging and communication in medicine (DICOM) server or the like immediately after the image is captured by the modality, and automatically executing available processing for the image is necessary. At this point, in a case where a processing request is made for all automatically acquired images satisfying an automatic execution condition of image processing, the following problems arise.

[Problem 1] For example, a processing request is sent to a destination (processing request destination) for sending the processing request, such as the image processing API loaded on premises or on the cloud, from a terminal on a medical institution side. However, in a case where all processing requests satisfying the automatic execution condition are sent regardless of insufficient calculation resources (resources) at the processing request destination, the resources of the processing request destination are exhausted by calculation for obtaining a processing result of low priority, and there is a possibility that processing of high priority is not executed well. Here, the processing result of low priority refers to, for example, an image processing result that is not frequently used in the actual diagnostic workflow.

[Problem 2] For example, in sending a processing request on a side on which the processing request is sent, sending the processing request by setting priority such as high/medium/low to cause the processing request destination to perform processing of high priority first is also considered with respect to Problem 1. However, determining optimal priority on a user side so that the diagnostic workflow of a user is smoothly finished is difficult. In addition, it is also assumed that three or more steps of granularity of the priority are necessary for flexible priority setting, and determining the priority is even more difficult for the user. Thus, a system for systematically optimizing the priority is desired.

[Problem 3] In a case where an amount of resources of an on-premises server or a cloud server on a side on which the processing request is received is reduced to be less than usual because of a system failure and the like, there is a possibility of significant deterioration in usability such that unless adjustment such as further reducing the number of processing requests sent from the processing request side to be less than usual compared to a usual time is performed, a necessary processing result is not returned even after a long time. Adjustment similar to the above is also necessary in a case where a rate limit of the API is set to protect resources on an image processing API side.

That is, in a case where processing requests are continuously sent from a client terminal side regardless of a load situation on a server side on which image processing is performed, an operation on the server side may be unstable, or a long waiting time may occur even for acquiring a minimum necessary processing result on the client terminal side.

The present disclosure is conceived in view of such a matter, and an object thereof is to solve at least one of the plurality of problems and to provide a medical image processing system, a medical image processing method, an information processing apparatus, and a program that can secure stability of a system and usability. The present disclosure suggests a system for effectively narrowing down the number of processing requests by dynamically determining a priority order of requested processing in sending a processing request on a client terminal side on which the processing request is automatically made for a new image captured by a modality, and for returning a minimum necessary processing result (processing result of high priority) on the client terminal side as quickly as possible.

A medical image processing system according to an aspect of the present disclosure is a medical image processing system comprising an image processing server that performs image processing on a medical image, and an information processing apparatus connected to the image processing server through a network, in which the image processing server includes one or more first processors, the first processor is configured to execute a plurality of processing modules for performing a plurality of types of image processing, and receive an image of a processing target and a processing request from the information processing apparatus, execute image processing corresponding to the processing request, and return a processing result to a request source, the information processing apparatus includes one or more second processors, and the second processor is configured to collect an operating log of an image viewer used in a case where a user views the processing result of the image processing on a medical institution internal network to which the information processing apparatus is connected, calculate priority of each of the plurality of types of image processing based on the collected operating log, record information about the priority obtained by the calculation and update and manage priority information of each of the plurality of types of image processing, acquire a new image captured by one or more modalities connected to the medical institution internal network, determine which image processing is executable for the acquired new image among the plurality of types of image processing, perceive a load situation of the image processing server, and transmit, based on the priority of each of one or more types of the determined executable image processing and on the perceived load situation of the image processing server, a processing request of the one or more types of the executable image processing to the image processing server in accordance with a criterion of the priority.

According to the medical image processing system of the present aspect, in transmitting the processing request of the image processing for the acquired image in the information processing apparatus on a side on which the processing request is output, the number of processing requests transmitted to the image processing server (the number of transmitted processing requests) is controlled by perceiving the load situation of the image processing server and determining processing to be actually requested to the image processing server by considering the priority of each executable image processing for the image of the target and the load situation of the image processing server.

Accordingly, the processing request of processing of high priority is preferentially performed in accordance with the load situation, and the processing request of processing of low priority is suppressed in a situation where resources of the image processing server are insufficient. Since the priority of each image processing is calculated based on the operating log in a case where a user has referred to the processing result and the like using the image viewer, the processing result of high necessity for the user or the processing result having a high priority order can be appropriately determined. According to the present aspect, even in a situation where the resources of the image processing server are insufficient, the processing result of high priority can be acquired relatively early, and stability and/or responsiveness as a whole system can be secured.

In the medical image processing system according to another aspect of the present disclosure, the image processing server may be installed on the network accessible from the information processing apparatus of each of a plurality of medical institutions.

The medical image processing system according to another aspect of the present disclosure may further comprise a plurality of the information processing apparatuses connected to the image processing server through the network, in which the plurality of information processing apparatuses include terminals connected to medical institution internal networks of medical institutions different from each other, respectively.

In the medical image processing system according to another aspect of the present disclosure, an image storage server that stores the image captured by the one or more modalities may be installed on the medical institution internal network.

In the medical image processing system according to another aspect of the present disclosure, the information processing apparatus may acquire the number of times reference is made to the processing result of the image processing and information about a reference order from the operating log, and calculate the priority of each image processing using the number of times reference is made and the information about the reference order.

In the medical image processing system according to another aspect of the present disclosure, the information processing apparatus may double as the image viewer.

In the medical image processing system according to another aspect of the present disclosure, a plurality of the image viewers may be connected to the medical institution internal network, and the information processing apparatus may collect the operating log of each of the plurality of image viewers, and calculate the priority by statistically processing information recorded in a plurality of the collected operating logs.

In the medical image processing system according to another aspect of the present disclosure, the information processing apparatus may perform organ extraction processing of extracting an organ captured in the acquired new image, and determine the image processing related to the organ as the executable image processing among the plurality of types of image processing based on information about the extracted organ.

In the medical image processing system according to another aspect of the present disclosure, the information processing apparatus may determine the executable image processing from the plurality of types of image processing based on tag information attached to the acquired new image.

In the medical image processing system according to another aspect of the present disclosure, the image processing server may include an endpoint that receives an inquiry about the load situation from the information processing apparatus and that responds with a current load situation, and the information processing apparatus may acquire information indicating the load situation of the image processing server from the endpoint by using the endpoint.

In the medical image processing system according to another aspect of the present disclosure, the information processing apparatus may perceive the load situation of the image processing server by recording a response time taken from the transmission of the processing request to the image processing server to obtaining of the processing result for each processing request, and calculating a rate of increase in the response time.

In the medical image processing system according to another aspect of the present disclosure, the information processing apparatus may compare a numerical value indicating the perceived load situation of the image processing server with a threshold value, and transmit a processing request of processing of the priority corresponding to the threshold value to the image processing server.

In the medical image processing system according to another aspect of the present disclosure, the priority may be divided into levels of 50 or more steps from a level of lowest priority to a level of highest priority.

In the medical image processing system according to another aspect of the present disclosure, the plurality of types of image processing may include at least one processing of computer aided detection (CADe) processing or computer aided diagnosis (CADx) processing.

In the medical image processing system according to another aspect of the present disclosure, the plurality of processing modules may include a CADe module for performing the CADe processing, and a CADx module for performing the CADx processing.

In the medical image processing system according to another aspect of the present disclosure, in a default setting, priority of the CADe processing may be set to priority higher than priority of the CADx processing.

In the medical image processing system according to another aspect of the present disclosure, the plurality of processing modules may include a processing module for performing report creation support processing including processing of generating a candidate for a medical opinion.

In the medical image processing system according to another aspect of the present disclosure, in a default setting, priority of the report creation support processing may be set to priority lower than priority of the CADe processing and lower than priority of the CADx processing.

In the medical image processing system according to another aspect of the present disclosure, the plurality of types of image processing may include at least one processing of fracture detection processing of detecting a position of a fracture, bone labeling processing of labeling a bone number, lung nodule detection processing of detecting a position of a lung nodule, characteristic identification processing of identifying a characteristic of the lung nodule, or lung section labeling processing of labeling a lung section.

A medical image processing method according to another aspect of the present disclosure is a medical image processing method for transmitting an image of a processing target and a processing request to an image processing server capable of performing a plurality of types of image processing from an information processing apparatus connected to the image processing server through a network, and for executing image processing corresponding to the processing request and returning a processing result to a request source in the image processing server, the medical image processing method comprising collecting an operating log of an image viewer used in a case where a user views the processing result of the image processing on a medical institution internal network to which the information processing apparatus is connected, calculating priority of each of the plurality of types of image processing based on the collected operating log, recording information about the priority obtained by the calculation and updating and managing priority information of each of the plurality of types of image processing, acquiring a new image captured by one or more modalities connected to the medical institution internal network, determining which image processing is executable for the acquired new image among the plurality of types of image processing, perceiving a load situation of the image processing server, and making, based on the priority of each of one or more types of the determined executable image processing and on the perceived load situation of the image processing server, a processing request of the one or more types of the executable image processing to the image processing server in accordance with a criterion of the priority.

An information processing apparatus according to another aspect of the present disclosure is an information processing apparatus connected to an image processing server capable of performing a plurality of types of image processing through a network, the information processing apparatus comprising one or more processors, in which the processor is configured to collect an operating log of an image viewer used in a case where a user views a processing result of the image processing on a medical institution internal network to which the information processing apparatus is connected, calculate priority of each of the plurality of types of image processing based on the collected operating log, record information about the priority obtained by the calculation and update and manage priority information of each of the plurality of types of image processing, acquire a new image captured by one or more modalities connected to the medical institution internal network, determine which image processing is executable for the acquired new image among the plurality of types of image processing, perceive a load situation of the image processing server, and transmit, based on the priority of each of one or more types of the determined executable image processing and on the perceived load situation of the image processing server, a processing request of the one or more types of the executable image processing to the image processing server in accordance with a criterion of the priority.

A program according to another aspect of the present disclosure is a program causing a computer to function as an information processing apparatus connected to an image processing server capable of performing a plurality of types of image processing through a network, the program causing the computer to implement a function of collecting an operating log of an image viewer used in a case where a user views a processing result of the image processing on a medical institution internal network to which the information processing apparatus is connected, a function of calculating priority of each of the plurality of types of image processing based on the collected operating log, a function of recording information about the priority obtained by the calculation and updating and managing priority information of each of the plurality of types of image processing, a function of acquiring a new image captured by one or more modalities connected to the medical institution internal network, a function of determining which image processing is executable for the acquired new image among the plurality of types of image processing, a function of perceiving a load situation of the image processing server, and a function of transmitting, based on the priority of each of one or more types of the determined executable image processing and on the perceived load situation of the image processing server, a processing request of the one or more types of the executable image processing to the image processing server in accordance with a criterion of the priority.

According to the present invention, in the information processing apparatus on the side on which the processing request of the image processing is made, the number of processing requests transmitted to the image processing server can be effectively narrowed down based on the load situation of the image processing server and on the priority of each image processing. According to the present invention, stability of an operation of the image processing server providing the processing result of the image processing corresponding to the processing request can be secured. In addition, the information processing apparatus according to the present invention can quickly acquire the processing result of high necessity for the user, and usability is secured.

### BRIEF DESCRIPTION OF THE DRAWINGS

FIG. 1 is a block diagram schematically illustrating a configuration and an operation of a medical image processing system according to an embodiment of the present invention.

FIG. 2 is a flowchart illustrating a flow of operation of the medical image processing system illustrated in FIG. 1.

FIG. 3 is a flowchart illustrating an example of an operation related to priority calculation.

FIG. 4 is a diagram schematically illustrating a system configuration example of the medical image processing system.

FIG. 5 is a block diagram illustrating a configuration example of an image processing API server.

FIG. 6 is a block diagram illustrating a configuration example of an image processing management terminal on a medical institution internal network.

FIG. 7 is a block diagram illustrating a configuration example of a viewer terminal.

FIG. 8 is a block diagram illustrating an example of a hardware configuration of a computer.

### DESCRIPTION OF THE PREFERRED EMBODIMENTS

Hereinafter, a preferred embodiment of the present invention will be described in accordance with the appended drawings.

<<Summary of Medical Image Processing System>>

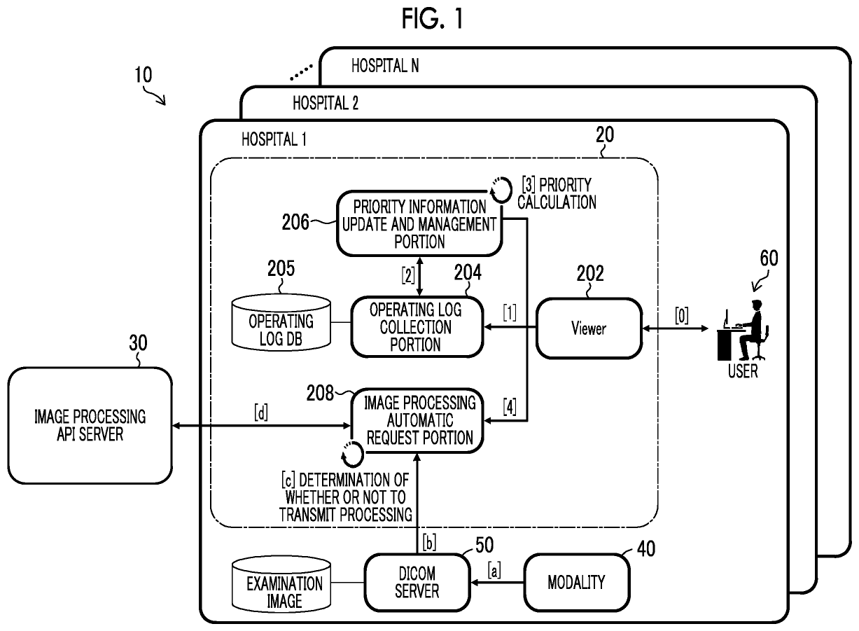

FIG. 1 is a block diagram schematically illustrating a configuration and an operation of a medical image processing system 10 according to an embodiment of the present invention. The medical image processing system 10 includes a terminal 20 installed on a medical institution internal network of each of a plurality of medical institutions and an image processing API server 30 installed on a network accessible from the terminal 20 of each medical institution.

Here, the terminal 20 refers to a calculation resource present in a network in which data inside the medical institution can be safely accessed. The terminal 20 may not be physically present inside the medical institution. The terminal 20 of each medical institution may be a physical machine or a virtual machine, and a specific form thereof is not limited. A representative example of the medical institution is a “hospital”. Display of “hospital 1”, “hospital 2”, . . . , “hospital N” illustrated in FIG. 1 represents that N medical institutions are present. At least one terminal 20 is provided on the medical institution internal network for one medical institution. The terminal 20 is an example of an “information processing apparatus” according to the embodiment of the present disclosure.

The image processing API server 30 is a central image processing server that receives a processing request of image processing from the terminal 20 of each of the N medical institutions, executes requested image processing, and returns a processing result to a request source.

One or more modalities 40 and a DICOM server 50 are connected to the medical institution internal network of each medical institution. The modality 40 is an apparatus that captures an examination image. The modality 40 includes an apparatus that images an examination target part of a subject to generate an examination image representing the part and that outputs the image by adding accessory information defined by the DICOM standard to the image. Specific examples of the modality 40 include a computed tomography (CT) apparatus (computer tomography apparatus), a magnetic resonance imaging (MRI) apparatus (magnetic resonance image capturing apparatus), an angiography X-ray diagnostic apparatus, a positron emission tomography (PET) apparatus (positive electron emission tomography apparatus), an ultrasonic apparatus, a computed radiography (CR) apparatus (computer X-ray imaging apparatus) using a flat X-ray detector (flat panel detector (FPD)), a mammography apparatus, and an endoscope apparatus.

The DICOM server 50 is a server that operates in accordance with DICOM specifications. The DICOM server 50 is a computer that stores and manages various types of data including the image captured using the modality 40 and that comprises a high-capacity external storage device and a database management program. The DICOM server 50 is an example of an “image storage server” according to the embodiment of the present disclosure.

Each of a viewer 202, an operating log collection portion 204, a priority information update and management portion 206, and an image processing automatic request portion 208 is constructed on one or more terminals 20 in each medical institution. The viewer 202 includes a radiographic interpretation viewer program for supporting a diagnostic workflow of image diagnosis by a doctor. The viewer 202 displays the examination image, the image processing result, and the like on a display device. The viewer 202 may be dedicated viewing software or a web browser or the like. The viewer 202 is an example of an “image viewer” according to the embodiment of the present disclosure.

The operating log collection portion 204 is a program for collecting an operating log in a case where a user 60 has used the viewer 202. The operating log collection portion 204 automatically collects the operating log at an appropriate time without causing the user 60 to be aware of collection work of the operating log. Data of the collected operating log is stored in an operating log database (DB) 205.

The user 60 is mainly a doctor or the like and is a person who refers to the image processing result by using the viewer 202.

The priority information update and management portion 206 is a program for calculating a priority order of various types of medical image processing based on the operating log collected by the operating log collection portion 204. The priority information update and management portion 206 may calculate the priority order for each individual based on the operating log acquired for each individual of the user 60 or may calculate the priority order on an assumption of average usage in the medical institution by statistically processing the operating logs of a plurality of the users 60.

The image processing automatic request portion 208 is a program for acquiring the image stored in the DICOM server 50, selecting image processing to be applied to the acquired image, and requesting the image processing API server 30 to perform the image processing in accordance with priority information set by the priority information update and management portion 206.

Establishing connection between the image processing API server 30 and the image processing automatic request portion 208 present on the medical institution internal network of each medical institution enables mutual communication.

The image processing API server 30 may be in any form of a physical machine, a virtual machine, or the like as long as the image processing API server 30 is present on a network safely accessible from the image processing automatic request portion 208 on the medical institution internal network of each of the plurality of medical institutions. The image processing API server 30 may be a cloud server or an on-premises server. The image processing API server 30 is an example of an “image processing server” according to the embodiment of the present disclosure.

While an example in which the viewer 202, the operating log collection portion 204, the priority information update and management portion 206, and the image processing automatic request portion 208 are constructed on one terminal 20 is illustrated in FIG. 1, these programs may be distributedly constructed over two or more terminals present on the medical institution internal network. For example, the viewer 202 may be constructed on a first terminal, and the operating log collection portion 204, the priority information update and management portion 206, and the image processing automatic request portion 208 may be constructed on a second terminal different from the first terminal. Alternatively, each of the viewer 202, the operating log collection portion 204, the priority information update and management portion 206, and the image processing automatic request portion 208 may be constructed on a separate terminal.

A flow of procedures [a] to [d] in FIG. 1 illustrates a flow from the capturing of the examination image by the modality 40 to the processing request made to the image processing API server 30. A flow of procedures [0] to [4] in FIG. 1 illustrates a flow of calculating the priority of various types of image processing from the operating log of the usage of the viewer 202 by the user 60. The flow of the procedures [a] to [d] and the flow of the procedures [0] to [4] may be simultaneously performed in parallel.

<<Description of Medical Image Processing Method>>

A specific example of how the user 60 uses the medical image processing system 10 will be described below using the procedure [0] and the flow of the procedures [a] to [d] in FIG. 1 as an example.

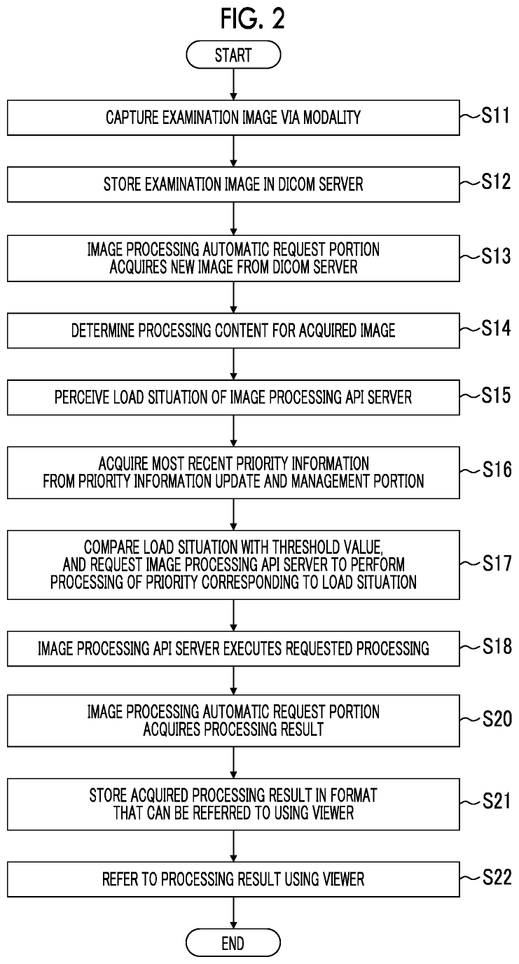

FIG. 2 is a flowchart illustrating a flow of operation of the medical image processing system 10. First, the examination image is captured by the modality 40 (step S11). The examination image captured by the modality 40 is stored in the DICOM server 50 (step S12; procedure [a] in FIG. 1).

Next, the image processing automatic request portion 208 automatically acquires the image newly stored in the DICOM server 50 (step S13; procedure [b] in FIG. 1), and processing content of which processing may be performed on the image acquired by the image processing automatic request portion 208 is determined (step S14; procedure [c] in FIG. 1). Here, “automatically” includes a meaning that an input of an instruction provided by an operation from the user 60 at each acquisition is not necessary, and means automatic acquisition in the background in conjunction with an operation of storing the image captured by the modality 40 in the DICOM server 50.

Here, as a system for determining which processing may be performed on the image acquired by the image processing automatic request portion 208, for example, the image processing automatic request portion 208 may extract information about which organ is captured by performing organ extraction processing on the acquired image and, in a case where a lung is captured, determine that lung nodule detection is to be executed. One or more types of executable image processing are linked to each organ, and one or more types of executable image processing are determined in accordance with the extracted organ. A plurality of types of executable image processing may be present for one image.

In addition, the image processing automatic request portion 208 may use information obtained from a DICOM tag attached to the image as a processing applicability determination criterion. For example, a condition such as a CT slice thickness may be used as the information obtained from the DICOM tag. In a case where the CT slice thickness is large, processing that cannot be executed may be present. Accordingly, a condition that processing can be performed in a case where the CT slice thickness is less than or equal to a predetermined criterion value may be set, and processing applicability may be determined based on the information about the CT slice thickness. Of course, the processing applicability may be determined based on a combination of a result of the organ extraction and the condition of the CT slice thickness.

In addition, the determination of the processing applicability may also be executed from only the DICOM tag. For example, in a case where capturing using a contrast agent is perceived from the DICOM tag, a determination that processing of performing angiography or extracting a lesion and/or an abnormality in a blood vessel is to be executed can be made. The information about the DICOM tag is an example of “tag information” according to the embodiment of the present disclosure.

Next, the image processing automatic request portion 208 performs an operation for perceiving a load situation of the image processing API server 30, before transmitting a request of processing determined as being applicable to the acquired image to the image processing API server 30 (step S15).

Here, as a method of perceiving the load situation, for example, an API endpoint that responds with a load situation on an image processing API server 30 side, such as an API endpoint that returns the number of types of processing in a waiting state in the current image processing API server 30, may be comprised on the image processing API server 30 side, and the image processing automatic request portion 208 may use the API endpoint to acquire the load situation of the image processing API server 30 from response content. The API endpoint is an example of an “endpoint” according to the embodiment of the present disclosure.

In addition, as another example of the method of perceiving the load situation, a method in which the image processing automatic request portion 208 perceives the load situation by recording a response time taken so far from the transmission of the processing request to the image processing API server 30 to obtaining of the processing result inside the image processing automatic request portion 208 for each processing request and by, for example, calculating a tendency of increase in time taken until the response is returned may be used.

Next, the image processing automatic request portion 208 acquires the most recent priority information of each processing from the priority information update and management portion 206 as needed (step S16; procedure [4] in FIG. 1).

The image processing automatic request portion 208 compares a numerical value indicating the load situation perceived in step S15 with a threshold value and requests the image processing API server 30 to perform processing of priority corresponding to the threshold value (step S17; procedure [d] in FIG. 1).

For example, the priority is divided into levels of 100 steps from “1” indicating the lowest level to “100” indicating the highest level, and the priority is determined for each processing by the priority information update and management portion 206. In this case, for example, if a rate of increase in the response time of the image processing API server 30 is 30% in the last 10 requests, it is possible for the image processing automatic request portion 208 to transmit the processing request of processing of which the priority is 30 to 100.

While an example of the priority of 1 to 100 is illustrated here as an example of granularity of the priority, a definition of the priority is not limited to this example. More detailed granularity of the priority is desired in order to flexibly set the priority order with respect to a plurality of types of image processing. For example, the priority is preferably divided into levels of 50 or more steps from a level of the lowest priority to a level of the highest priority and is further preferably divided into levels of 100 or more steps. For example, the levels of the priority may be defined in 256 steps or may be defined in 1024 steps.

The image processing API server 30 that has received the processing request from the image processing automatic request portion 208 executes the requested processing (step S18).

Then, the image processing automatic request portion 208 acquires the processing result from the image processing API server 30 at an appropriate time (step S20).

Here, in acquiring the processing result, a notification indicating that the processing result is created may be provided to the image processing automatic request portion 208 from the image processing API server 30. Alternatively, a flow in which the image processing automatic request portion 208 periodically issues an inquiry about presence or absence of the processing result to the image processing API server 30 and acquires the processing result in a case where the processing result is present may also be possible.

The image processing automatic request portion 208 that has acquired the processing result from the image processing API server 30 stores the processing result in a format that can be referred to using the viewer 202 (step S21). Information about the processing result may be stored in the DICOM server 50 in association with the image.

Then, the user 60 can refer to the processing result through the viewer 202 (step S22).

Here, in a case where calculation resources of the image processing API server 30 are sufficient for the processing request from the image processing automatic request portion 208, it is assumed that all processing results determined as being applicable to the image acquired from the DICOM server 50 by the image processing automatic request portion 208 in step S13 and step S14 can be referred to using the viewer 202 in a case where the user 60 refers to the processing result in the above procedure. However, in a case where processing requests transmitted from the image processing automatic request portion 208 are narrowed down as a result of considering the load situation of the image processing API server 30 in step S15, it is assumed that some of the results are not prepared when the user 60 refers to the processing result.

A specific example of such a case will be described below.

### Specific Example 1

For example, two types of processing including fracture CAD of performing fracture detection from the image and bone labeling of labeling bone numbers are considered. The fracture CAD is generally performed at the beginning of the diagnostic workflow in order to find a fracture. Thus, the number of times reference is made to a result of the fracture CAD using the viewer 202 is also large, and it is desirable that the processing result of the fracture CAD can be referred to at an early step on a user 60 side.

On the other hand, the bone labeling is, for example, processing of automatically recognizing an ordinal number of a vertebra. The labeling of the bone number obtained as a result of the bone labeling has an important role in specifying a fracture location on a report in a case where a fracture is detected by the fracture CAD. On the other hand, in a case where a fracture is not detected, the bone number does not need to be written on the report, and the processing of the bone labeling may not be performed. In addition, even in a case where a fracture is detected and the result of the bone labeling is not present, the report can be created by specifying the bone number by the user 60 while this may be inconvenient for the user 60 side. For this reason, a determination of “fracture CAD>bone labeling” can be made as the priority of image processing with respect to the present example.

A relative magnitude relationship of the priority may be set in setting (setting at a time of shipment) default priority or may be determined from an analysis result of the operating log of the viewer 202. For example, the default priority may be set, and then, preference and the like of the user 60 in each medical institution may be reflected on the priority from the analysis of the operating log.

Alternatively, transmitting each processing request to the image processing API server 30 without assigning any priority at first is also considered. In this case, it is assumed that it takes time until the processing result can be referred to, and the user 60 such as a doctor feels inconvenienced at first. However, the priority can be set for each processing such that processing that is waited for until the processing is finished is processing of high priority, and that processing that is canceled without waiting is processing of low priority. Then, processing on which the priority is reflected can be performed.

In a case where the relationship “fracture CAD>bone labeling” is present for the priority, the image processing automatic request portion 208 preferentially makes a processing request of the fracture CAD over the processing of the bone labeling.

### Specific Example 2

As another example, three types of processing including lung nodule detection, lung section labeling, and report candidate text generation processing using processing results of the two types are present. There is a possibility that a lung nodule indicates any disorder, and a result of the lung nodule detection of detecting a position of the lung nodule is frequently referred to at an early step of the diagnostic workflow.

On the other hand, the lung section labeling is a function of dividing a region of the lung into distinctive regions and is generally referred to later than the result of the lung nodule detection. In a case where abnormal findings are not seen in a lung field image, describing a name of a lung region in detail on the report may not be necessary, and a result of the lung section labeling may not be referred to.

Thus, the relationship of the priority between the lung nodule detection and the lung section labeling can be said to be “lung nodule detection>lung section labeling”. The report candidate text generation processing is processing of generating a candidate for a medical opinion to be described on the report. The report candidate text generation processing generates the candidate for the medical opinion using the result of the lung nodule detection and the result of the lung section labeling as an input. However, even in a case where the candidate for the medical opinion is not present, the report can be created in a case where the result of the lung nodule detection and the result of the lung section labeling are present, while this may be inconvenient for the user 60. From such a viewpoint, the relationship of the priority between the three types of processing can be determined as “lung nodule detection>lung section labeling>report candidate text generation”. In this case, the image processing automatic request portion 208 can perform processing such as preferentially making a processing request of the lung nodule detection. The report candidate text generation processing is an example of “report creation support processing” according to the embodiment of the present disclosure.

<Example of Determining Whether or Not to Transmit Processing Request>

In “Specific Example 1” and “Specific Example 2” described above, the idea of the priority of various types of medical image processing has been described using specific examples. In a case where the resources of the image processing API server 30 are insufficient, requests are transmitted to the image processing API server 30 from the image processing automatic request portion 208 in an order of fracture CAD→bone labeling in, for example, “Specific Example 1”. Here, a determination as to whether or not to transmit the processing request of the bone labeling after the fracture CAD is performed by the image processing automatic request portion 208 based on the following criterion.

[Rule 1] The image processing automatic request portion 208 periodically perceives the load situation of the image processing API server 30 in step S15.

[Rule 2] The image processing automatic request portion 208 internally holds a processing request (for example, the processing request of the bone labeling) that has to be transmitted but cannot be transmitted yet because of low priority.

[Rule 3] In a case where the threshold value of the load situation of the image processing API server 30 perceived in step S15 is compared with the priority of the held processing request (example: bone labeling), and a load situation in which the held processing request may be transmitted occurs, the held processing request is transmitted to the image processing API server 30.

Here, with respect to a case where the load situation of the image processing API server 30 is not improved even after an elapse of a certain time, and the held processing request cannot be easily transmitted, a timeout time may be provided so that transmission of the held processing request is canceled after an elapse of a certain time. A value of the timeout time may be provided as a fixed value as a setting file or the like. For example, the image processing automatic request portion 208 may calculate an average time taken for a radiographic interpretation workflow from the operating log collected by the operating log collection portion 204 and dynamically set the timeout time based on the calculated time. For example, in a case where one radiographic interpretation workflow is usually (averagely) finished in 30 minutes, and the held processing request is not transmitted even after an elapse of 60 minutes that is twice the average time, a result of the radiographic interpretation workflow may be considered to not be necessary anymore, and dynamic setting of setting 60 minutes as the timeout time may be performed.

<Example of Method of Calculating Priority>

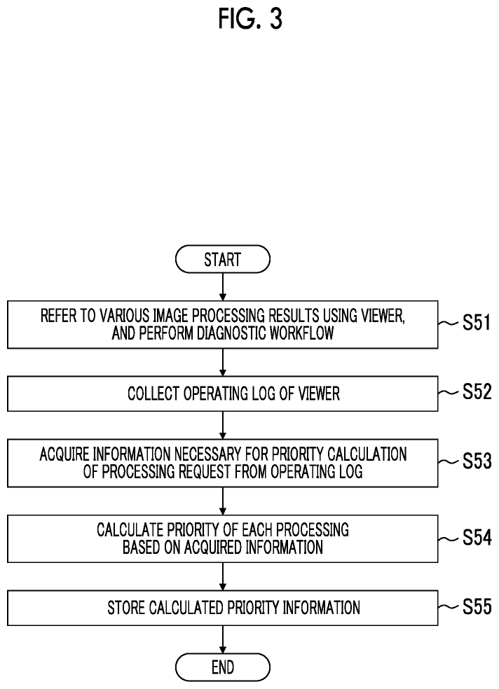

The idea of the priority of each processing has been described so far. Here, the flow of the procedures [0] to [4] in FIG. 1 will be described, and a specific example related to priority calculation will be illustrated. FIG. 3 is a flowchart illustrating an example of an operation related to the priority calculation.

In the procedure [0] in FIG. 1, first, the user 60 refers to various image processing results using the viewer 202 and performs the diagnostic workflow (step S51 in FIG. 3).

Next, in the procedure [1], the operating log collection portion 204 collects the operating log of the operation of the viewer 202 by the user 60 (step S52).

Next, in the procedure [2], the priority information update and management portion 206 acquires information necessary for the priority calculation of the processing request from the operating log (step S53). As the information necessary for the priority calculation, for example, the priority information update and management portion 206 acquires a log related to the number of times reference is made to the processing result, and information about a reference order such as which processing result is referred to after which processing result is referred to.

The priority information update and management portion 206 that has acquired the necessary information calculates the priority of each processing using the information in the procedure [3] (step S54). Processing here is processing of requesting the image processing API server 30 to perform, for example, the lung nodule detection from the image processing automatic request portion 208 in each medical institution. In the calculation of the priority, for example, the following priority criterion is applied.

[Priority Criterion] High priority is assigned to a processing result that is referred to first and for which the number of times reference is made is large in a number of diagnostic workflows.

This criterion is based on an idea that since the processing result is referred to first in the diagnostic workflow, the processing result needs to be quickly returned. Specific examples are described in “Specific Example 1” and “Specific Example 2” described above.

A subsequent method of calculating the priority may be provided as a setting file for the information processing apparatus in which the priority information update and management portion 206 is constructed, or may be directly implemented as a source code.

While the priority of processing of which the number of times reference is made to the result thereof is large is set to be high in accordance with the “Priority Criterion” described above, it is also preferable to consider the following criterion as a priority criterion other than the number of times reference is made. That is, it is considered that the priority of processing necessary as a pre-step of another processing, such as processing of a CADe system that is processing in a pre-step of processing of a CADx system, also needs to be set to be high. The processing of the CADx system is processing of performing characteristic analysis. For example, processing of determining (identifying) cancer, pneumonia, or the like corresponds to the processing of the CADx system. On the other hand, the processing of the CADe system is processing of a detection system for detecting a specific region or a target from the image. For example, processing of detecting whether an abnormal region is present on the image and extracting the abnormal region corresponds to the processing of the CADe system. A stepwise processing aspect in which the characteristic analysis is performed by the processing of the CADx system in a case where an abnormal region is detected by the processing of the CADe system is considered.

In order to handle such a situation, for example, a default priority value other than a value of the number of times reference is made to the result may be assigned to each processing as an attribute for the value of the priority of various types of processing, and the number of times reference is made to the result may be provided as a value to be added to the default value.

For example, priority of 200 as an attribute is provided for the processing of the CADe system as the default priority value, and priority of 100 as an attribute is provided for the processing of the CADx system. The number of times, specified from the operating log, reference is made to the result is provided as a value to be added to the default value. Accordingly, the value of the priority in which the default priority value and the actual number of times reference is made is dynamically set.

In addition, for example, in a case where certain processing A of the CADe system having priority of 200 is necessary for three types of processing B, processing C, and processing D of the CADx system having priority of 100 in a subsequent step, a calculation rule of the priority such that the priority of processing of which the processing result is necessary for another processing is set to be high, such as setting the priority of the processing A to 200+100×3=500, may be provided. For example, the processing A may be abnormal region extraction processing in the lung. For example, the processing B may be lung cancer AI determination processing. For example, the processing C may be pneumonia AI determination processing. For example, the processing D may be bronchitis AI determination processing. The abnormal region extraction processing in the lung is an example of “CADe processing” according to the embodiment of the present disclosure. Each of the lung cancer AI determination processing, the pneumonia AI determination processing, and the bronchitis AI determination processing is an example of “CADx processing” according to the embodiment of the present disclosure.

The priority information calculated in step S54 (procedure [3] in FIG. 1) is stored in the priority information update and management portion 206 (step S55). The image processing automatic request portion 208 acquires the most recent priority information of each processing before transmitting the processing request of image processing at a time such as step S16 and step S17 described using FIG. 2.

<<System Configuration Example>>

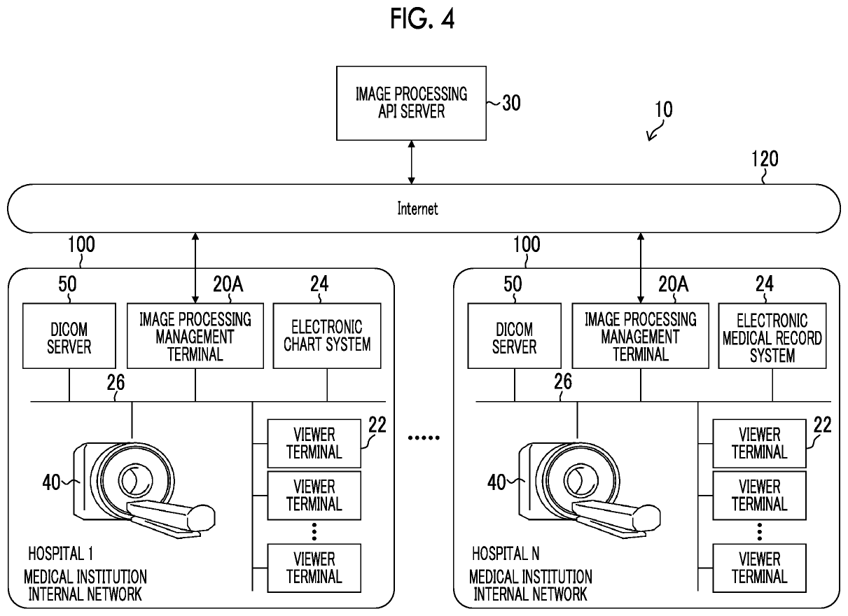

Next, an example of a specific configuration of the medical image processing system 10 will be described. FIG. 4 is a diagram schematically illustrating a system configuration example of the medical image processing system 10. First, an example of a medical institution internal network 100 will be described. In FIG. 4, while an example in which the medical institution internal network 100 having the same system configuration is constructed in each of the plurality of medical institutions is illustrated for simplification of illustration, a medical institution internal network having different system configurations for each medical institution may be constructed.

The medical institution internal network 100 is a computer network including the modality 40, the DICOM server 50, an image processing management terminal 20A, a viewer terminal 22, an electronic medical record system 24, and an on-premises communication line 26.

The medical institution internal network 100 may include a plurality of types of the modalities 40. Various combinations of the types of modalities 40 connected to the medical institution internal network 100 are possible for each medical institution.

The DICOM server 50 communicates with other apparatuses through the on-premises communication line 26 and transmits and receives various types of data including image data. The DICOM server 50 receives image data generated by the modality 40 and other various types of data via the on-premises communication line 26 and manages the data by storing the data in a recording medium such as a high-capacity external storage device. A storage format of the image data and communication between each apparatus via the on-premises communication line 26 are based on a DICOM protocol.

The image processing management terminal 20A is an information processing apparatus corresponding to the terminal 20 described using FIG. 1. A form of the image processing management terminal 20A is not particularly limited and may be a personal computer, a workstation, a tablet terminal, or the like. The image processing management terminal 20A has a communication function for communicating with the image processing API server 30 and is connected to the image processing API server 30 through a wide-area communication line 120. The image processing management terminal 20A can acquire data from the DICOM server 50 and the like through the on-premises communication line 26. In addition, the image processing management terminal 20A can transmit the processing result acquired from the image processing API server 30 to the DICOM server 50 and to the viewer terminal 22. The image processing management terminal 20A may double as the viewer terminal 22.

Various types of data stored in a database of the DICOM server 50 and various types of information including the processing result acquired by the image processing management terminal 20A can be displayed on the viewer terminal 22.

The viewer terminal 22 is an image viewing terminal called a PACS viewer or a DICOM viewer. A plurality of the viewer terminals 22 may be connected to the medical institution internal network 100. A form of the viewer terminal 22 is not particularly limited and may be a personal computer, a workstation, a tablet terminal, or the like.

As illustrated in FIG. 4, the medical institution internal network having the same system configuration is constructed in each of the plurality of medical institutions.

The image processing API server 30 communicates with the image processing management terminal 20A of each medical institution through the wide-area communication line 120. The wide-area communication line 120 is an example of a “network” according to the embodiment of the present disclosure. The image processing API server 30 can perform a plurality of types of image processing and provides various image processing services in response to the processing request from the image processing management terminal 20A.

For example, image processing provided by the image processing API server 30 may include at least one processing of fracture detection processing of detecting a position of a fracture, bone labeling processing of labeling a bone number, lung nodule detection processing of detecting a position of a lung nodule, lung nodule characteristic identification processing of identifying a characteristic of a lung nodule, or lung section labeling processing of labeling a lung section. In addition, image processing provided by the image processing API server 30 may include organ segmentation processing, blood vessel region extraction processing, brain CAD processing, mammary gland CAD processing, liver CAD processing, colon CAD processing, report creation support processing, and the like.

<<Configuration Example of Image Processing API Server 30>>

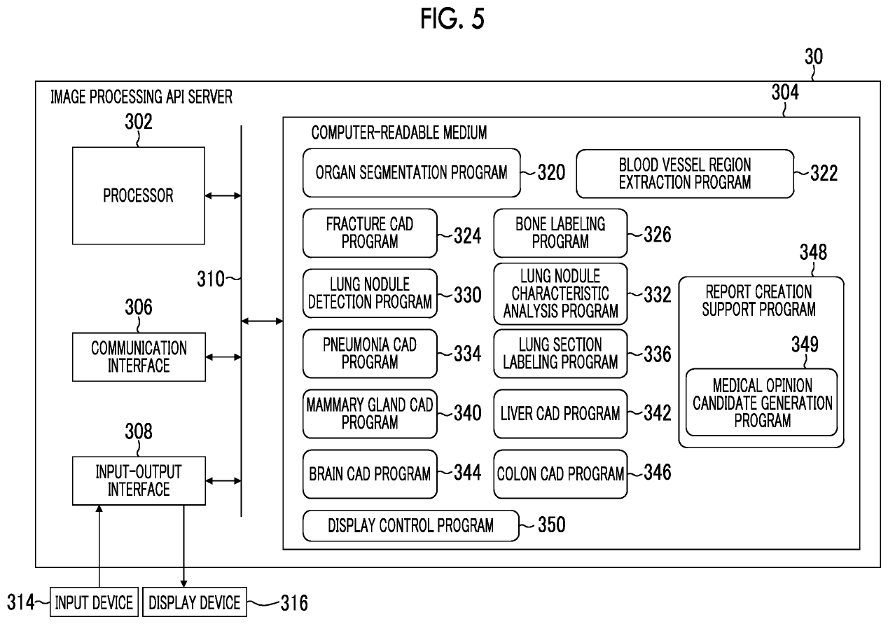

FIG. 5 is a block diagram illustrating a configuration example of the image processing API server 30. The image processing API server 30 can be implemented by a computer system configured using one or a plurality of computers. Various functions of the image processing API server 30 are implemented by installing a program on the computer.

The image processing API server 30 comprises a processor 302, a non-transitory tangible computer-readable medium 304, a communication interface 306, an input-output interface 308, a bus 310, an input device 314, and a display device 316. The processor 302 is an example of a “first processor” according to the embodiment of the present disclosure. The computer-readable medium 304 is an example of a “first storage device” according to the embodiment of the present disclosure.

The processor 302 includes a central processing unit (CPU). The processor 302 may include a graphics processing unit (GPU). The processor 302 is connected to the computer-readable medium 304, the communication interface 306, and the input-output interface 308 through the bus 310. The input device 314 and the display device 316 are connected to the bus 310 through the input-output interface 308.

The computer-readable medium 304 includes a memory that is a main memory, and a storage that is an auxiliary storage device. For example, the computer-readable medium 304 may be a semiconductor memory, a hard disk drive (HDD) device, a solid state drive (SSD) device, or a combination of a plurality thereof.

The image processing API server 30 is connected to the wide-area communication line 120 (refer to FIG. 4) through the communication interface 306.

The computer-readable medium 304 stores a plurality of programs for performing various types of processing including the plurality of types of image processing, data, and the like. For example, the computer-readable medium 304 may store one or more programs of an organ segmentation program 320, a blood vessel region extraction program 322, a fracture CAD program 324, a bone labeling program 326, a lung nodule detection program 330, a lung nodule characteristic analysis program 332, a pneumonia CAD program 334, a lung section labeling program 336, a mammary gland CAD program 340, a liver CAD program 342, a brain CAD program 344, a colon CAD program 346, and a report creation support program 348, and the like. The report creation support program 348 includes a medical opinion candidate generation program 349. These various processing programs may be AI processing modules including a trained model that is trained to obtain an output of a target task by applying machine learning such as deep learning.

For example, an AI model for CAD can be configured using various convolutional neural networks (CNNs) having a convolutional layer. For example, input data for the AI model may include a medical image such as a two-dimensional image, a three-dimensional image, or a motion picture image, and an output from the AI model may be information indicating a position of a disease region (lesion part) in the image, information indicating a classification such as a disease name, or a combination thereof.

For example, an AI model that handles time series data, document data, and the like can be configured using various recurrent neural networks (RNNs). The time series data includes, for example, waveform data of an electrocardiogram. The document data includes, for example, a medical opinion created by a doctor.

Each processing program illustrated in FIG. 5 is an example of a “processing module” according to the embodiment of the present disclosure. Each of the fracture CAD program 324 and the lung nodule detection program 330 is an example of a “CADe module” according to the embodiment of the present disclosure. Each of the lung nodule characteristic analysis program 332 and the pneumonia CAD program 334 is an example of a “CADx module” according to the embodiment of the present disclosure. Various forms of types and combinations of processing programs implemented in the image processing API server 30 are possible. A server program including various processing programs stored in the computer-readable medium 304 is an example of a “first program” according to the embodiment of the present disclosure.

By executing instructions of the processing programs via the processor 302, the computer of the image processing API server 30 functions as processing units corresponding to the processing programs. For example, by executing an instruction of the organ segmentation program 320 via the processor 302, the computer of the image processing API server 30 functions as an organ segmentation processing unit that performs the organ segmentation processing. The same applies to the other programs.

By executing the instructions of the programs stored in the computer-readable medium 304, the processor 302 performs an operation of receiving the image of the processing target and the processing request from the image processing management terminal 20A of each medical institution, executing image processing corresponding to the processing request, and returning the processing result to the request source. The operation executed by the image processing API server 30 is an example of a “first operation” according to the embodiment of the present disclosure.

In addition, the computer-readable medium 304 stores a display control program 350. The display control program 350 generates a display signal necessary for a display output to the display device 316 and performs a display control of the display device 316.

For example, the display device 316 is composed of a liquid crystal display, an organic electro-luminescence (OEL) display, a projector, or an appropriate combination thereof. For example, the input device 314 is composed of a keyboard, a mouse, a touch panel, other pointing devices, a voice input device, or an appropriate combination thereof. The input device 314 receives various inputs from an operator. The display device 316 and the input device 314 may be configured to be integrated using a touch panel.

<<Configuration Example of Image Processing Management Terminal 20A>>

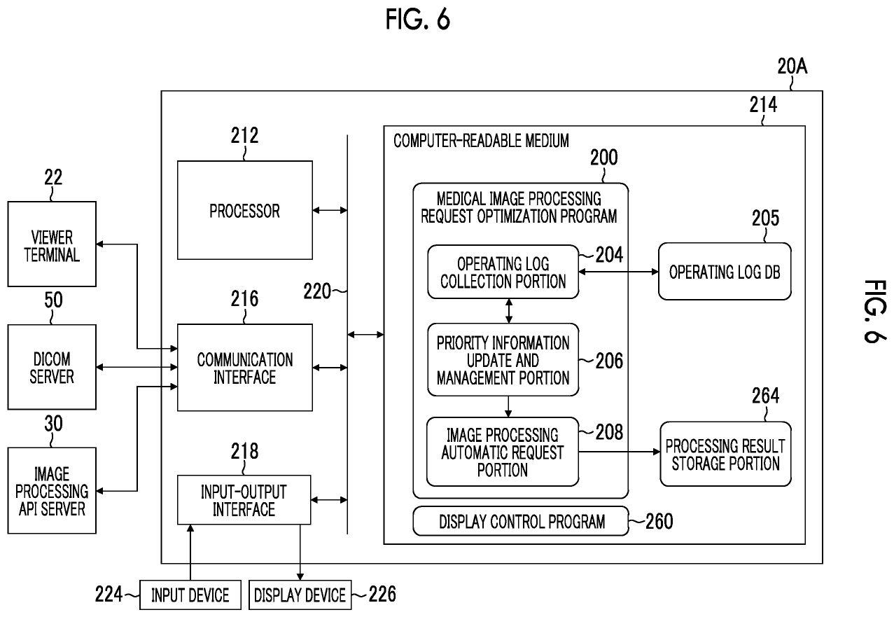

FIG. 6 is a block diagram illustrating a configuration example of the image processing management terminal 20A on the medical institution internal network 100. The image processing management terminal 20A can be implemented by a computer system configured using one or a plurality of computers.

The image processing management terminal 20A comprises a processor 212, a non-transitory tangible computer-readable medium 214, a communication interface 216, an input-output interface 218, a bus 220, an input device 224, and a display device 226. A hardware configuration of the image processing management terminal 20A may be the same as the hardware configuration of the image processing API server 30 described using FIG. 5. That is, a hardware configuration of each of the processor 212, the computer-readable medium 214, the communication interface 216, the input-output interface 218, the bus 220, the input device 224, and the display device 226 illustrated in FIG. 6 may be the same as the corresponding element thereof illustrated in FIG. 5.

The processor 212 is an example of a “second processor” and a “processor” according to the embodiment of the present disclosure. The computer-readable medium 214 is an example of a “second storage device” and a “storage device” according to the embodiment of the present disclosure.

The image processing management terminal 20A is connected to the viewer terminal 22, the DICOM server 50, and the image processing API server 30 through the communication interface 216.

The computer-readable medium 214 stores various programs including a medical image processing request optimization program 200 and a display control program 260 and data. The medical image processing request optimization program 200 includes the operating log collection portion 204, the priority information update and management portion 206, and the image processing automatic request portion 208. In addition, the computer-readable medium 214 includes an operating log database 205 in which the data of the operating log collected by the operating log collection portion 204 is stored and managed, and a processing result storage portion 264 that stores the image processing result acquired from the image processing API server 30 by the image processing automatic request portion 208. The display control program 260 generates a display signal necessary for a display output to the display device 226 and performs a display control of the display device 226.

By executing an instruction of the medical image processing request optimization program 200 stored in the computer-readable medium 214, the processor 212 performs an operation including collecting the operating log of the viewer terminal 22, calculating the priority of each image processing based on the collected operating log, recording the information about the priority obtained by the calculating and updating and managing the priority information of each image processing, acquiring a new image captured by the modality 40, determining which image processing can be executed for the acquired new image, perceiving the load situation of the image processing API server 30, and transmitting the processing request to the image processing API server 30 in accordance with the criterion of the priority based on the priority of each determined executable image processing and on the load situation of the image processing API server 30. The operation executed by the image processing management terminal 20A is an example of a “second operation” according to the embodiment of the present disclosure.

The medical image processing request optimization program 200 is an example of a “second program” and a “program” according to the embodiment of the present disclosure. Here, while an example of constructing the operating log collection portion 204, the priority information update and management portion 206, and the image processing automatic request portion 208 in one image processing management terminal 20A is illustrated, processing functions of the medical image processing request optimization program 200 may be implemented by distributing the processing functions over a plurality of more than two computers.

<<Configuration Example of Viewer Terminal 22>>

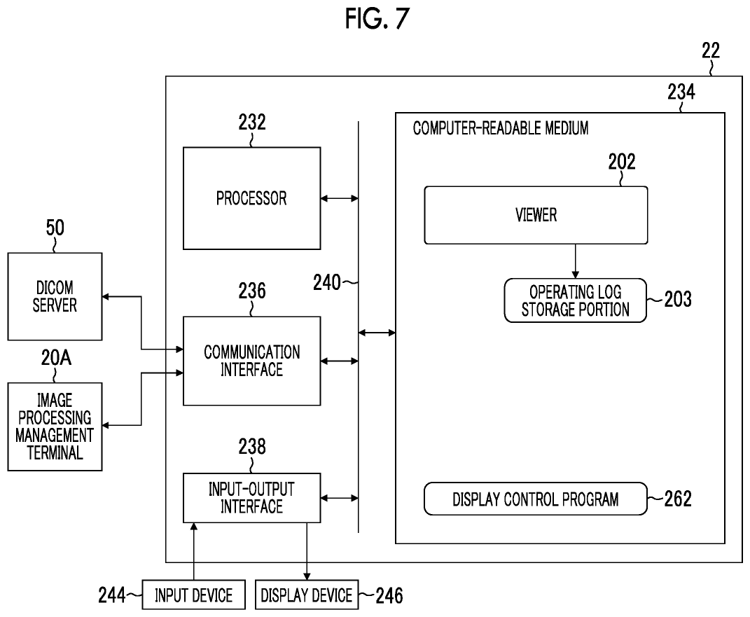

FIG. 7 is a block diagram illustrating a configuration example of the viewer terminal 22. A hardware configuration of the viewer terminal 22 may be the same as the hardware configuration of the image processing management terminal 20A described using FIG. 6. The viewer terminal 22 comprises a processor 232, a computer-readable medium 234, a communication interface 236, an input-output interface 238, a bus 240, an input device 244, and a display device 246. Each hardware configuration may be the same as the corresponding element thereof in the configuration illustrated in FIG. 6.

The computer-readable medium 234 includes the viewer 202 that is a medical image viewing program, an operating log storage portion 203 that stores the operating log of the viewer 202, and the display control program 262.

The viewer 202 displays various types of information including the image read out from the DICOM server 50 connected through the communication interface 236 and the processing result of image processing on the display device 246. In addition, the viewer 202 stores a history (operating log) of operation of the input device 244 by the user 60 in the operating log storage portion 203. Data of the operating log stored in the operating log storage portion 203 is transmitted to the operating log collection portion 204 of the image processing management terminal 20A. The display control program 262 generates a display signal necessary for a display output to the display device 246 and performs a display control of the display device 246.

<<Example of Hardware Configuration of Computer>>

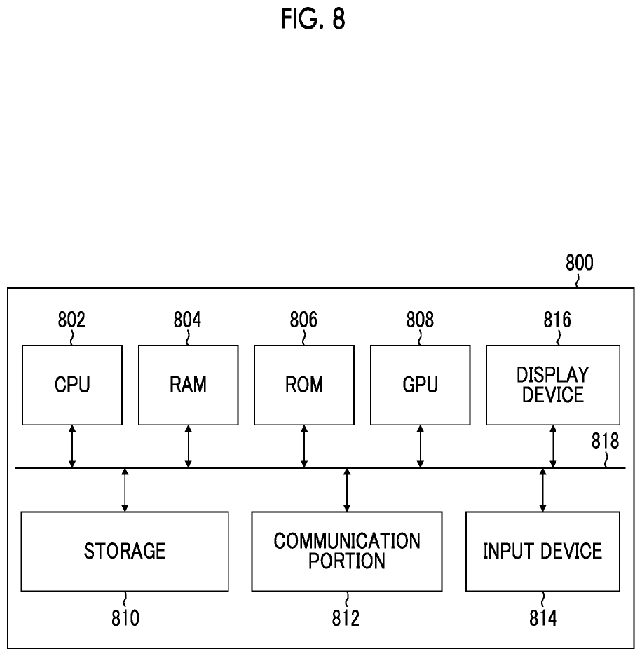

FIG. 8 is a block diagram illustrating an example of a hardware configuration of the computer. A computer 800 may be a personal computer, a workstation, or a server computer. The computer 800 can be used as an apparatus that comprises a part or all of the terminal 20, the image processing API server 30, the DICOM server 50, the electronic medical record system 24, the image processing management terminal 20A, and the viewer terminal 22 described above, or that has a plurality of functions thereof.

The computer 800 comprises a CPU 802, a random access memory (RAM) 804, a read only memory (ROM) 806, a GPU 808, a storage 810, a communication portion 812, an input device 814, a display device 816, and a bus 818. The GPU 808 may be provided as needed.

The CPU 802 reads out various programs stored in the ROM 806, the storage 810, or the like and executes various types of processing. The RAM 804 is used as a work region of the CPU 802. In addition, the RAM 804 is used as a storage unit that transitorily stores the read-out programs and various types of data.

For example, the storage 810 is configured to include a hard disk apparatus, an optical disc, a magneto-optical disk, a semiconductor memory, or a storage device configured using an appropriate combination thereof. The storage 810 stores various programs, data, and the like. By loading the programs stored in the storage 810 into the RAM 804 and executing the programs via the CPU 802, the computer 800 functions as a unit that performs various types of processing defined by the programs.

The communication portion 812 is an interface for performing communication processing with an external apparatus in a wired or wireless manner and exchanging information with the external apparatus. The communication portion 812 can have a role as an information acquisition portion that receives an input of the image and the like.

The input device 814 is an input interface for receiving various operation inputs for the computer 800. For example, the input device 814 may be a keyboard, a mouse, a touch panel, other pointing devices, a voice input device, or an appropriate combination thereof.

The display device 816 is an output interface on which various types of information are displayed. For example, the display device 816 may be a liquid crystal display, an organic electro-luminescence (OEL) display, a projector, or an appropriate combination thereof

<<Program for Operating Computer>>

A program that causes the computer to implement a part or all of at least one processing function of various processing functions such as an operating log collection function, a priority information update and management function, and an image processing automatic request function in the terminal 20 and in the image processing management terminal 20A and various image processing functions in the image processing API server 30 described in the embodiment can be recorded on a computer-readable medium that is an optical disc, a magnetic disk, a semiconductor memory, or another non-transitory tangible information storage medium, and the program can be provided through the information storage medium.

In addition, instead of an aspect of providing the program by storing the program in the non-transitory tangible computer-readable medium, a program signal can be provided as a download service by using an electric communication line such as the Internet.

<<Hardware Configuration of Each Processing Unit>>

For example, a hardware structure of a processing unit executing various types of processing of the operating log collection portion 204, the priority information update and management portion 206, the image processing automatic request portion 208, and the like in the terminal 20 and in the image processing management terminal 20A corresponds to the following various processors.

The various processors include a CPU that is a general-purpose processor functioning as various processing units by executing a program, a GPU that is a processor specialized in image processing, a programmable logic device (PLD) such as a field programmable gate array (FPGA) that is a processor of which a circuit configuration can be changed after manufacture, a dedicated electric circuit such as an application specific integrated circuit (ASIC) that is a processor having a circuit configuration dedicatedly designed to execute specific processing, and the like.

One processing unit may be composed of one of the various processors or may be composed of two or more processors of the same type or different types. For example, one processing unit may be composed of a plurality of FPGAs, a combination of a CPU and an FPGA, or a combination of a CPU and a GPU. In addition, a plurality of processing units may be composed of one processor. Examples of the plurality of processing units composed of one processor include, first, as represented by a computer such as a client or a server, a form in which one processor is composed of a combination of one or more CPUs and software, and this processor functions as the plurality of processing units. Second, as represented by a system on chip (SoC) or the like, a form of using a processor that implements functions of the whole system including the plurality of processing units via one integrated circuit (IC) chip is included. Accordingly, various processing units are configured using one or more of the various processors as a hardware structure.

Furthermore, the hardware structure of the various processors is more specifically an electric circuit (circuitry) in which circuit elements such as semiconductor elements are combined.

### Advantages of Present Embodiment

According to the medical image processing system 10 according to the embodiment of the present invention, the following advantages are achieved.

[1] In a case where a new image is captured by the modality 40, the new image is automatically acquired by the image processing automatic request portion 208, and the processing request of necessary image processing is automatically made in accordance with the load situation of the image processing API server 30. In a situation where the resources of the image processing API server 30 are insufficient, the processing request of processing of low priority is suppressed, and processing requests are transmitted by narrowing down the processing requests to processing of high necessity (high priority) for the user 60. Thus, it is possible to secure stability of the whole system while securing usability.

[2] Since the priority of each image processing is calculated based on the operating log in a case where the user has referred to the processing result and the like using the viewer 202, the processing result of high necessity for the user or the processing result having a high priority order can be appropriately determined. In addition, since the priority is calculated at an appropriate time and the priority information is updated in the priority information update and management portion 206, complicated work such as priority setting work by the user 60 or the like is not necessary.

[3] According to the medical image processing system 10 according to the present embodiment, since the image processing result of high necessity for the user 60 is in a viewable state in a short time after the image is captured by the modality 40, diagnostic work can be efficiently performed.

### Other

Configurations of the embodiment of the present invention described above can be appropriately changed, added, or removed without departing from the gist of the present invention. The present invention is not limited to the embodiment described above and can be subjected to many modifications by those having ordinary knowledge in the field within the technical idea of the present invention.

### EXPLANATION OF REFERENCES

10: medical image processing system
20: terminal
20A: image processing management terminal
22: viewer terminal
24: electronic medical record system
26: on-premises communication line
30: image processing API server
40: modality
50: DICOM server
60: user
100: medical institution internal network
120: wide-area communication line
200: medical image processing request optimization program
202: viewer
203: operating log storage portion
204: operating log collection portion
205: operating log database
206: priority information update and management portion
208: image processing automatic request portion
212: processor
214: computer-readable medium
216: communication interface
218: input-output interface
220: bus
224: input device
226: display device
232: processor
234: computer-readable medium
236: communication interface
238: input-output interface
240: bus
244: input device
246: display device
260: display control program
262: display control program
264: processing result storage portion
302: processor
304: computer-readable medium
306: communication interface
308: input-output interface
310: bus
314: input device
316: display device
320: organ segmentation program
322: blood vessel region extraction program
324: fracture CAD program
326: bone labeling program
330: lung nodule detection program
332: lung nodule characteristic analysis program
334: pneumonia CAD program
336: lung section labeling program
340: mammary gland CAD program
342: liver CAD program
344: brain CAD program
346: colon CAD program
348: report creation support program
349: medical opinion candidate generation program
350: display control program
800: computer
802: CPU
804: RAM
806: ROM
808: GPU
810: storage
812: communication portion
814: input device
816: display device
818: bus
S11 to S22: step of illustrating operation of medical image processing system
S51 to S55: step of processing of calculation priority

What is claimed is:

1. A medical image processing system comprising:
an image processing server that performs image processing on a medical image; and
an information processing apparatus connected to the image processing server through a network,
wherein the image processing server includes one or more first processors,
the first processor is configured to:
execute a plurality of processing modules for performing a plurality of types of image processing; and
receive an image of a processing target and a processing request from the information processing apparatus, execute image processing corresponding to the processing request, and return a processing result to a request source,
the information processing apparatus includes one or more second processors, and
the second processor is configured to:
collect an operating log of an image viewer used in a case where a user views the processing result of the image processing on a medical institution internal network to which the information processing apparatus is connected;
calculate priority of each of the plurality of types of image processing based on the collected operating log;
record information about the priority obtained by the calculation and update and manage priority information of each of the plurality of types of image processing;
acquire a new image captured by one or more modalities connected to the medical institution internal network;
determine which image processing is executable for the acquired new image among the plurality of types of image processing;
perceive a load situation of the image processing server; and
transmit, based on the priority of each of one or more types of the determined executable image processing and on the perceived load situation of the image processing server, a processing request of the one or more types of the executable image processing to the image processing server in accordance with a criterion of the priority.

2. The medical image processing system according to claim 1,
wherein the image processing server is installed on the network accessible from the information processing apparatus of each of a plurality of medical institutions.

3. The medical image processing system according to claim 1, further comprising:
a plurality of the information processing apparatuses connected to the image processing server through the network,
wherein the plurality of information processing apparatuses include terminals connected to medical institution internal networks of medical institutions different from each other, respectively.

4. The medical image processing system according to claim 1,
wherein an image storage server that stores the image captured by the one or more modalities is installed on the medical institution internal network.

5. The medical image processing system according to claim 1,
wherein the information processing apparatus acquires the number of times reference is made to the processing result of the image processing and information about a reference order from the operating log, and calculates the priority of each image processing using the number of times reference is made and the information about the reference order.

6. The medical image processing system according to claim 1,
wherein the information processing apparatus doubles as the image viewer.

7. The medical image processing system according to claim 1,
wherein a plurality of the image viewers are connected to the medical institution internal network, and
the information processing apparatus
collects the operating log of each of the plurality of image viewers, and
calculates the priority by statistically processing information recorded in a plurality of the collected operating logs.

8. The medical image processing system according to claim 1,
wherein the information processing apparatus
performs organ extraction processing of extracting an organ captured in the acquired new image, and
determines the image processing related to the organ as the executable image processing among the plurality of types of image processing based on information about the extracted organ.

9. The medical image processing system according to claim 1,
wherein the information processing apparatus determines the executable image processing from the plurality of types of image processing based on tag information attached to the acquired new image.

10. The medical image processing system according to claim 1,
wherein the image processing server includes an endpoint that receives an inquiry about the load situation from the information processing apparatus and that responds with a current load situation, and
the information processing apparatus acquires information indicating the load situation of the image processing server from the endpoint by using the endpoint.

11. The medical image processing system according to claim 1,
wherein the information processing apparatus perceives the load situation of the image processing server by recording a response time taken from the transmission of the processing request to the image processing server to obtaining of the processing result for each processing request, and calculating a rate of increase in the response time.

12. The medical image processing system according to claim 1,
wherein the information processing apparatus
compares a numerical value indicating the perceived load situation of the image processing server with a threshold value, and
transmits a processing request of processing of the priority corresponding to the threshold value to the image processing server.

13. The medical image processing system according to claim 1,
wherein the priority is divided into levels of 50 or more steps from a level of lowest priority to a level of highest priority.

14. The medical image processing system according to claim 1,
wherein the plurality of types of image processing include at least one processing of computer aided detection (CADe) processing or computer aided diagnosis (CADx) processing.

15. The medical image processing system according to claim 14,
wherein the plurality of processing modules include
a CADe module for performing the CADe processing, and
a CADx module for performing the CADx processing.

16. The medical image processing system according to claim 15,
wherein in a default setting, priority of the CADe processing is set to priority higher than priority of the CADx processing.

17. The medical image processing system according to claim 14,
wherein the plurality of processing modules include a processing module for performing report creation support processing including processing of generating a candidate for a medical opinion.

18. The medical image processing system according to claim 17,
wherein in a default setting, priority of the report creation support processing is set to priority lower than priority of the CADe processing and lower than priority of the CADx processing.

19. The medical image processing system according to claim 1,
wherein the plurality of types of image processing include at least one processing of fracture detection processing of detecting a position of a fracture,
bone labeling processing of labeling a bone number,
lung nodule detection processing of detecting a position of a lung nodule,
characteristic identification processing of identifying a characteristic of the lung nodule, or
lung section labeling processing of labeling a lung section.

20. A medical image processing method for transmitting an image of a processing target and a processing request to an image processing server capable of performing a plurality of types of image processing from an information processing apparatus connected to the image processing server through a network, and for executing image processing corresponding to the processing request and returning a processing result to a request source in the image processing server, the medical image processing method comprising:
collecting an operating log of an image viewer used in a case where a user views the processing result of the image processing on a medical institution internal network to which the information processing apparatus is connected;
calculating priority of each of the plurality of types of image processing based on the collected operating log;
recording information about the priority obtained by the calculation and updating and managing priority information of each of the plurality of types of image processing;
acquiring a new image captured by one or more modalities connected to the medical institution internal network;
determining which image processing is executable for the acquired new image among the plurality of types of image processing;
perceiving a load situation of the image processing server; and
making, based on the priority of each of one or more types of the determined executable image processing and on the perceived load situation of the image processing server, a processing request of the one or more types of the executable image processing to the image processing server in accordance with a criterion of the priority.

21. An information processing apparatus connected to an image processing server capable of performing a plurality of types of image processing through a network, the information processing apparatus comprising:
one or more processors,
wherein the processor is configured to:
collect an operating log of an image viewer used in a case where a user views a processing result of the image processing on a medical institution internal network to which the information processing apparatus is connected;
calculate priority of each of the plurality of types of image processing based on the collected operating log;
record information about the priority obtained by the calculation and update and manage priority information of each of the plurality of types of image processing;
acquire a new image captured by one or more modalities connected to the medical institution internal network;
determine which image processing is executable for the acquired new image among the plurality of types of image processing;
perceive a load situation of the image processing server; and
transmit, based on the priority of each of one or more types of the determined executable image processing and on the perceived load situation of the image processing server, a processing request of the one or more types of the executable image processing to the image processing server in accordance with a criterion of the priority.

22. A non-transitory, computer-readable tangible recording medium which records thereon, a program for causing, when read by a computer, the computer to function as an information processing apparatus connected to an image processing server capable of performing a plurality of types of image processing through a network, the program causing the computer to implement:
a function of collecting an operating log of an image viewer used in a case where a user views a processing result of the image processing on a medical institution internal network to which the information processing apparatus is connected;
a function of calculating priority of each of the plurality of types of image processing based on the collected operating log;
a function of recording information about the priority obtained by the calculation and updating and managing priority information of each of the plurality of types of image processing;
a function of acquiring a new image captured by one or more modalities connected to the medical institution internal network;
a function of determining which image processing is executable for the acquired new image among the plurality of types of image processing;
a function of perceiving a load situation of the image processing server; and
a function of transmitting, based on the priority of each of one or more types of the determined executable image processing and on the perceived load situation of the image processing server, a processing request of the one or more types of the executable image processing to the image processing server in accordance with a criterion of the priority.
